(19) United States
(12) Patent Application Publication
(10) Pub. No.: US 2026/0337495 A1
(43) Pub. Date: Oct. 29, 2026

(54) METHODS FOR DEHYDROGENATING HYDROCARBONS BY THERMAL DEHYDROGENATION

(71) Applicant: Dow Global Technologies LLC, Midland, MI (US)

(72) Inventors: Matthew T. Pretz, Lake Jackson, TX (US); Barry B. Fish, Lake Jackson, TX (US); Eric E. Stangland, Midland, MI (US); Brian W. Goodfellow, Freeport, TX (US); Manish Sharma, Lake Jackson, TX (US)

(73) Assignee: Dow Global Technologies LLC, Midland, MI (US)

(21) Appl. No.: 19/111,471

(22) PCT Filed: Sep. 12, 2023
(86) PCT No.: PCT/US2023/073963

§ 371 Date: Mar. 13, 2025

Related U.S. Application Data
(60) Provisional application No. 63/406,449, filed on Sep. 14, 2022.

(51) Int. Cl. C10G 9/32

(52) U.S. Cl. CPC C10G 9/32; C10G 2300/1048; C10G 2300/1081; C10G 2300/1096; C10G 2300/301

(57) ABSTRACT

A method for dehydrogenating hydrocarbons may include passing a hydrocarbon feed including one or more alkanes or alkyl aromatics into a fluidized bed reactor. In the fluidized bed reactor at least 95 wt. % of the hydrocarbon feed may have an atmospheric boiling point of less than or equal to 300 °C. The method may include thermally cracking the hydrocarbon feed in the fluidized bed reactor to produce a dehydrogenated product and hydrogen. The fluidized bed reactor may operate at a temperature of at least 600 °C. The fluidized bed reactor may be free of dehydrogenation catalyst. The method may include contacting the hydrogen with an oxygen-carrier material in the fluidized bed reactor to combust hydrogen and form an oxygen-diminished oxygen-carrier material. The oxygen-carrier material may be reducible.

### CROSS REFERENCE TO RELATED APPLICATION

This application claims priority to U.S. Provisional Application Ser. No. 63/406,449, filed Sep. 14, 2022, which is hereby incorporated by reference in its entirety.

### TECHNICAL FIELD

Embodiments described herein generally relate to chemical processing and, more specifically, to processes and systems utilized for dehydrogenation.

### BACKGROUND

Light olefins and aromatic olefins may be utilized as base materials to produce many types of goods and materials. For example, ethylene may be utilized to manufacture polyethylene, ethylene chloride, or ethylene oxides. Additionally, styrene may be used for the production of polystyrene. Such products may be utilized in product packaging, construction, textiles, etc. These base chemicals may be formed by dehydrogenation of hydrocarbon feeds. Thus, there is an industry demand for new dehydrogenation processes to form materials such as ethylene, propylene, butene, and styrene.

### SUMMARY

One method for producing light olefins and/or aromatic olefins is by thermal dehydrogenation of feed streams that include one or more alkanes, such as ethane, propane, n-butane, and/or i-butane, or alkyl aromatics such as ethylbenzene. By thermal dehydrogenation, chemicals may be dehydrogenated without the use of a dehydrogenation catalyst. Such a thermal dehydrogenation reaction produces hydrogen. According to the embodiments disclosed herein, this hydrogen, formed by thermal (i.e., non-catalytic) dehydrogenation, is reacted with oxygen from an oxygen-carrier material to form water, which can be separated from the product olefins. Moreover, while the thermal dehydrogenation reaction requires a relatively large heat input to the system, the reaction of the hydrogen with the oxygen from the oxygen-carrier material is exothermic and can, thus, offset at least a portion of the heat input load needed for the thermal dehydrogenation. The oxygen-carrier material may be cycled to a regeneration unit where it is replenished with oxygen, which may be exothermic and may offset some addition heat input into the system. Thus, methods described herein may efficiently produce light olefins without the need for a dehydrogenation catalyst.

Moreover, as described herein, embodiments may include the combustion of supplemental fuel in the regeneration unit. It has been discovered that process that fail to utilize supplemental fuels may not properly include a heat balance sufficient to maintain thermal dehydrogenation. Advantageously, the combustion of such supplemental fuels can be performed in the same area as the oxidation of the oxygen-carrier material (each using oxygen present in the regeneration unit). The heat from combustion of the supplemental fuel can raise the temperature of the oxygen-carrier material in the regeneration unit, which may be the main source of heat transport into the reactor where thermal dehydrogenation takes place.

According to at least one embodiment of the present disclosure, a method for dehydrogenating hydrocarbons may include passing a hydrocarbon feed comprising one or more alkanes or alkyl aromatics into a fluidized bed reactor. In the fluidized bed reactor at least 95 wt. % of the hydrocarbon feed may have an atmospheric boiling point of less than or equal to 300 °C. The method may further include thermally cracking the hydrocarbon feed in the fluidized bed reactor to produce a dehydrogenated product and hydrogen. The fluidized bed reactor may operate at a temperature of at least 600 °C. The fluidized bed reactor may be free of dehydrogenation catalyst. The method may further include contacting the hydrogen with an oxygen-carrier material in the fluidized bed reactor to combust the hydrogen and form an oxygen-diminished oxygen-carrier material. The oxygen-carrier material may be reducible. The method may further comprise passing the oxygen-diminished oxygen-carrier material to a regeneration unit, oxidizing the oxygen-diminished oxygen-carrier material in the regeneration unit to form the oxygen-rich oxygen-carrier material, combusting a supplemental fuel in the regeneration unit to produce heat and increase the temperature of the oxygen-carrier material, and passing the oxygen-rich oxygen-carrier material to the fluidized bed reactor.

These and other embodiments are described in more detail in the following Detailed Description in conjunction with the appended drawings.

### BRIEF DESCRIPTION OF THE DRAWINGS

The following detailed description of specific embodiments of the present disclosure can be best understood when read in conjunction with the following drawings, where like structure is indicated with like reference numerals and in which:

FIG. 1 schematically depicts a catalytic dehydrogenation system, according to one or more embodiments described herein; and

FIG. 2 schematically depicts a catalytic dehydrogenation system with recycle, according to one or more embodiments described herein.

It should be understood that the drawings are schematic in nature, and do not include some components of a reactor system commonly employed in the art, such as, without limitation, temperature transmitters, pressure transmitters, flow meters, pumps, valves, and the like. It would be known that these components are within the spirit and scope of the present embodiments disclosed. However, operational components, such as those described in the present disclosure, may be added to the embodiments described in this disclosure.

Reference will now be made in greater detail to various embodiments, some embodiments of which are illustrated in the accompanying drawings. Whenever possible, the same reference numerals will be used throughout the drawings to refer to the same or similar parts.

### DETAILED DESCRIPTION

Specific embodiments of the present application will now be described. The disclosure may, however, be embodied in different forms and should not be construed as limited to the embodiments set forth in this disclosure.

Embodiments related to methods for processing chemical streams to form products by thermal dehydrogenation are disclosed herein. In various embodiments, processes may include the use of thermal dehydrogenation along with an oxygen-carrier material. The methods may include thermally dehydrogenating an alkane or alkyl aromatic to form hydrogen and an olefin or olefinic aromatic, and then combusting that hydrogen with oxygen from the oxygen-carrier material as described herein.

As described herein, the processes do not include dehydrogenation catalysts. A dehydrogenation catalyst includes materials that catalyze the dehydrogenation reaction. To the extent that an oxygen-carrier material may only minimally catalyze the dehydrogenation reaction, the oxygen-carrier materials are not considered to be dehydrogenation catalysts as contemplated herein. That is, the oxygen-carrier materials utilized herein have very little or no catalytic functionality as compared to their activity in introducing oxygen.

There are many advantages of the utilization of processes that do not include dehydrogenation catalyst. For example, added costs of dehydrogenation catalysts, which may often need to be replaced or rejuvenated, are eliminated. Moreover, in one or more embodiments, the complexity of the reaction system is decreased since catalytic reactions are eliminated and, thus, only thermal dehydrogenation and combustion of hydrogen need to be accounted for in design.

Unless specified herein, an “oxygen-carrier material” may generally refer to an oxygen-rich oxygen-carrier material or an oxygen-deficient oxygen-carrier material. An oxygen-deficient state may be present after some oxygen is released and utilized for combustion of hydrogen, and may be oxygen-rich prior to the combustion of the hydrogen. Generally, the oxygen-carrier material may enter the regeneration unit in an oxygen-deficient state and leave the regeneration unit in an oxygen-rich state. The reactions that convert the oxygen-carrier material from an oxygen-rich state to an oxygen-deficient state may take place in one or more fluidized bed reactors, such as circulating fluidized bed reactors. The reactors may be, for example, risers or downers. It should be understood that the oxygen-rich state of the oxygen-carrier material may not be fully oxidized, and that the oxygen-deficient state of the oxygen-carrier may still include some releasable oxygen. However, the oxygen content of the oxygen-carrier material in the oxygen-rich state is generally greater than that of the oxygen-deficient state.

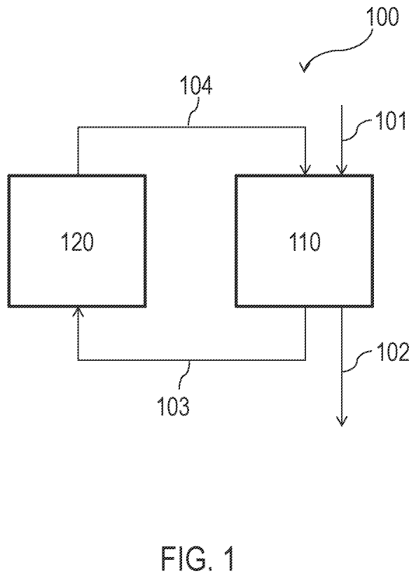

Now referring to FIG. 1, a reactor system 100 is depicted that may be used to perform the methods of the present disclosure. The reactor system 100 may include a fluidized bed reactor 110 and a regeneration unit 120. A hydrocarbon feed 101 may be passed into a fluidized bed reactor 110. The oxygen-carrier material may circulate between the fluidized bed reactor 110 and a regeneration unit 120 via streams 103 and 104, as depicted. The products may pass out of the fluidized bed reactor 110 via stream 102.

In one or more embodiments, the hydrocarbon feed 101 may comprise one or more alkanes or alkyl aromatics. For example, the hydrocarbon feed 101 may comprise one or more of ethane, propane, butane, or ethylbenzene. According to one or more embodiments, the hydrocarbon feed 101 may comprise at least 50 wt. %, at least 60 wt. %, at least 70 wt. %, at least 80 wt. %, at least 90 wt. %, at least 95 wt. % or even at least 99 wt. % of ethane. In additional embodiments, the hydrocarbon feed 101 may comprise at least 50 wt. %, at least 60 wt. %, at least 70 wt. %, at least 80 wt. %, at least 90 wt. %, at least 95 wt. % or even at least 99 wt. % of propane. In additional embodiments, the hydrocarbon feed 101 may comprise at least 50 wt. %, at least 60 wt. %, at least 70 wt. %, at least 80 wt. %, at least 90 wt. %, at least 95 wt. % or even at least 99 wt. % of butane. In additional embodiments, the hydrocarbon feed 101 may comprise at least 50 wt. %, at least 60 wt. %, at least 70 wt. %, at least 80 wt. %, at least 90 wt. %, at least 95 wt. % or even at least 99 wt. % of ethylbenzene. In additional embodiments, the hydrocarbon feed 101 may comprise at least 50 wt. %, at least 60 wt. %, at least 70 wt. %, at least 80 wt. %, at least 90 wt. %, at least 95 wt. % or even at least 99 wt. % of the sum of ethane, propane, butane and ethylbenzene.

According to one or more embodiments, at least 95 wt. % of the hydrocarbon feed 101 may have an atmospheric boiling point of less than or equal to 300 °C. According to additional embodiments, at least 95 wt. % of the hydrocarbon feed 101 may have an atmospheric boiling point of less than or equal to 275 °C, less than or equal to 250 °C, less than or equal to 225 °C, less than or equal to 200 °C, less than or equal to 175 °C, less than or equal to 150 °C, less than or equal to 125 °C, or even less than or equal to 100 °C. For example, the hydrocarbon feed 101 may not be a crude oil or a heavy cut of crude oil. In additional embodiments, at least 99 wt. % of the hydrocarbon feed 101 may have an atmospheric boiling point of less than or equal to 300 °C.

Referring still to FIG. 1, reactor system 100 may include a fluidized bed reactor 110 and a regeneration unit 120, both of which may be fluid bed based and with the same or different fluidization regimes. According to some embodiments, the fluidized bed reactor 110 may operate in a “back-mixed” fashion where the feed hydrocarbons enter the fluidized bed reactor 110 as to closely approximate isothermal conditions. As such, the fluid velocity at this region may be low enough and the solids flux may be great enough such that a dense bed may form at or around where the hydrocarbons are injected. In some embodiments, the superficial velocity in this region may be from 3-80 ft/s, such as from 3-40 ft/s, or 10-30 ft/s. The solids flux in the reactor may be from 1-300 lb/ft²-s, such as from 40-200 lb/ft²-s, or from 60 -160 lb/ft²-s. The fluidized bed reactor 110 may include multiple diameters, and may include one or more frustums to increase or decrease solids and/or gaseous reactant velocity. The fluidized bed reactor 110 may operate with a gas residence time of from 0.1-10 seconds, such as from 0.5-6 seconds.

An embodiment of the general operation of a fluidized bed reactor 110 to conduct a continuous reaction will now be described. As used herein, the “solids” in the fluidized bed reactor 110 may include the oxygen-carrier material. In some embodiments, the fluidized bed reactor 110 may include from 1 wt. % to 100 wt. %, such as from 95 wt. % to 100 wt. %, of oxygen-carrier material based on the total weight of all solids in the fluidized bed reactor.

During operation of the fluidized bed reactor 110 of the reactor system 100, the hydrocarbon feed 101 may enter the fluidized bed reactor 110, and the product stream may exit the reactor system 100 via stream 102. According to one or more embodiments, the reactor system 100 may be operated by feeding a chemical feed (e.g., in a feed stream such as hydrocarbon feed 101) into the fluidized bed reactor 110.

According to one or more embodiments, an oxygen-rich oxygen-carrier material may also be fed into the fluidized bed reactor 110 from the regeneration unit 120 via stream 104. The chemical feed of stream 101 may contact the oxygen-rich oxygen-carrier material in the fluidized bed reactor 110. Each of the chemical feed and the oxygen-rich oxygen-carrier material may flow upwardly into and through the fluidized bed reactor 110 and produce a chemical product and an oxygen-diminished oxygen-carrier material.

According to embodiments, exposure of the feed to elevated temperatures in the fluidized bed reactor 110 may cause thermal dehydrogenation, forming olefinic chemicals and hydrogen gas. Additionally, within the fluidized bed reactor 110, the hydrogen may be contacted with the oxygen-rich oxygen-carrier material. The oxygen-rich oxygen-carrier material may be reducible, and the contacting of the oxygen-rich oxygen-carrier material with the hydrogen may combust the hydrogen and form an oxygen-diminished oxygen-carrier material.

In some embodiments, the chemical product and the oxygen-diminished oxygen-carrier material may be passed to a separation device in a separation section within the fluidized bed reactor 110. The oxygen-diminished oxygen-carrier material may be separated from the chemical product (and any unreacted feed) in the separation device within the fluidized bed reactor 110. The chemical product (along with unreacted feed) may then be transported out of the separation section of the fluidized bed reactor 110. For example, the separated vapors may be removed from the fluidized bed reactor 110 via a pipe at a gas outlet port of the separation section within the fluidized bed reactor 110. According to one or more embodiments, the separation device may be a cyclonic separation system. The cyclonic separation system may include two or more stages of cyclonic separation.

In one or more embodiments, the fluidized bed reactor 110 may operate with a residence time of the gases in the fluidized bed reactor 110 of less than 10 seconds (such less than 9 seconds, less than 8 seconds, less than 7 seconds, less than 6 seconds, less than 5 seconds, less than 4 seconds, or even less than 3 seconds). Thermal dehydrogenation rate may vary with temperature, so as the residence time may vary, as would be understood by those skilled in the art.

In one or more embodiments, the fluidized bed reactor 110 may operate at a temperature of greater than 600 °C and less than or equal to 800 °C. In some embodiments, the temperature in the fluidized bed reactor 110 may be from 625 °C or 650 °C to 770 °C. In other embodiments, the temperature in the fluidized bed reactor 110 may be from 700 °C to 750 °C. Without being bound by any particular theory, it is believed that too low of temperature (e.g., 600 °C or less) may limit the maximum conversion of the hydrocarbon due to equilibrium constraints. Too low of temperatures may also result in a slow rate of oxygen release from the oxygen-carrier material and low hydrogen combustion. On the other hand, high temperatures (e.g., greater than 800 °C) may result in thermal degradation of the desired products produced and may result in a lower product selectivity than is economically feasible. In some embodiments, the primary feed component(s) may be propane, ethylbenzene, and/or butane, and the fluidized bed reactor 110 may operate at a temperature of greater than 600 °C. In additional embodiments, the primary feed component may be ethane, and the fluidized bed reactor 110 may operate at a temperature of at least 625 °C.

In some embodiments, the fluidized bed reactor 110 may operate at a pressure of at least atmospheric pressure (about 14.7 psia). In some embodiments, the fluidized bed reactor 110 may operate at a pressure of about 500 psia. In other embodiments, the fluidized bed reactor 110 may operate at a pressure from about 4 psia to about 160 psia, from about 20 psia to about 100 psia, or from about 30 psia to about 60 psia. In some embodiments, the regeneration unit 120 may operate with a pressure of within 30 psi of the fluidized bed reactor 110.

In some embodiments, the hydrocarbon feed may contact the oxygen-rich oxygen-carrier material in an upstream reactor section of the fluidized bed reactor 110. Each of the chemical feed and the oxygen-rich oxygen-carrier material may flow upwardly into and through the downstream reactor section of the fluidized bed reactor 110 to produce a chemical product and an oxygen-diminished oxygen-carrier material, where hydrogen is formed by thermal dehydrogenation and the hydrogen is combusted by oxygen from the oxygen-carrier material to form the oxygen-diminished state of the oxygen-carrier material. In one or more embodiments, a feed distributor within the fluidized bed reactor 110 may be operable to dispense the hydrocarbon feed stream at all shroud distributor velocities from 200 ft/s to 50 ft/s. In such embodiments, various feed streams may be utilized while maintaining the desired reactor characteristics, such as operating as a fast fluidized, turbulent, or bubbling bed reactor in the upstream reactor section of the fluidized bed reactor 110 and as a dilute phase riser reactor in the downstream reactor section of the fluidized bed reactor 110. For example, suitable distributors are disclosed in U.S. Pat. No. 9,370,759, the teachings of which are incorporated herein by reference in their entirety. The chemical product and the oxygen-diminished oxygen-carrier material may be passed out of the downstream reactor section of the fluidized bed reactor 110 to the separation device within the fluidized bed reactor 110, where the oxygen-diminished oxygen-carrier material may be separated from the chemical product.

In additional embodiments, the weight hourly space velocity (WHSV) for the disclosed processes may range from 0.1 pound (lb) to 100 lb of chemical feed per hour (h) per lb of solids in the reactor (lb feed/h/lb solids). In some embodiments, where the fluidized bed reactor 110 comprises an upstream reactor section that operates as a fast fluidized, turbulent, or bubbling bed reactor and a downstream reactor section that operates as a dilute phase riser reactor, the superficial gas velocity may range therein from 2 ft/s (about 0.61 m/s) to 10 ft/s (about 3.05 m/s) in the upstream reactor section, and from 30 ft/s (about 9.14 m/s) to 70 ft/s (about 21.31 m/s) in the downstream reactor section. In additional embodiments, a reactor configuration that is fully of a riser-type may operate at a single high superficial gas velocity, for example, in some embodiments at least 30 ft/s (about 9.15 m/s) throughout.

The residence time of the solids in the fluidized bed reactor 110 may typically vary from 0.5 seconds (sec) to 240 sec. In other embodiments, the residence time of the solids may be from about 0.5 sec to about 200 sec, from about 0.5 sec to about 100 sec, from about 0.5 sec to about 50 sec, or about 0.5 sec to about 20 sec.

In additional embodiments, the ratio of the solids to the hydrocarbon feed 101 in the fluidized bed reactor 110 may range from 5 to 150 on a weight to weight (w/w) basis. In some embodiments, the ratio may range from 10 to 40, such as from 12 to 36, or from 12 to 24.

In additional embodiments, the flux of the solids (e.g., the oxygen-carrier material) may be from 1 pound per square foot-second (lb/ft²-s) (about 4.89 kg/m²-s) to 300 lb/ft²-s (to about 97.7 kg/m²-s), such as from 1-20 lb/ft²-s, in the upstream reactor section, and from 1 lb/ft²-s (about 48.9 kg/m² -s) to 300 lb/ft²-s (about 489 kg/m²-s), such as from 10-100 lb/ft²-s, in the downstream reactor section.

In one or more embodiments, the solids (e.g., the oxygen-carrier material) may include solid particulates that are capable of fluidization. In some embodiments, the solids may exhibit properties known in the industry as “Geldart A” properties. Solids may be classified as “Group A” or “Group B” according to D. Geldart, Gas Fluidization Technology, John Wiley & Sons (New York, 1986), 34-37; and D. Geldart, “Types of Gas Fluidization,” Powder Technol. 7(1973 ) 285-292, which are incorporated herein by reference in their entireties. In one or more embodiments, the oxygen-carrier material may exhibit properties known in the industry as “Geldart A” properties. In other embodiments, the oxygen-carrier material may exhibit properties known in the industry as “Geldart B” properties.

Group A is understood by those skilled in the art as representing an aeratable powder, having a bubble-free range of fluidization; a high bed expansion; a slow and linear deaeration rate; bubble properties that may include a predominance of splitting/recoalescing bubbles, with a maximum bubble size and large wake; high levels of solids mixing and gas backmixing, assuming equal U-Umf (U is the velocity of the carrier gas, and Umf is the minimum fluidization velocity, typically though not necessarily measured in meters per second, m/s, i.e., there is excess gas velocity); axisymmetric slug properties; and no spouting, except in very shallow beds. The properties listed tend to improve as the mean particle size decreases, assuming equal [symbol] p; or as the <45 micrometers (um) proportion is increased; or as pressure, temperature, viscosity, and density of the gas increase. In general, the particles may exhibit a small mean particle size and/or low particle density (<1.4 grams per cubic centimeter, g/cm 3), fluidize easily, with smooth fluidization at low gas velocities, and may exhibit controlled bubbling with small bubbles at higher gas velocities.

Group B is understood by those skilled in the art as representing a “sand-like” powder that starts bubbling at Umf; that exhibits moderate bed expansion; a fast deaeration; no limits on bubble size; moderate levels of solids mixing and gas backmixing, assuming equal U-Umf; both axisymmetric and asymmetric slugs; and spouting in only shallow beds. These properties tend to improve as mean particle size decreases, but particle size distribution and, with some uncertainty, pressure, temperature, viscosity, or density of gas seem to do little to improve them. In general, most of the particles having a particle size ( [symbol] p) of 40 μm< [symbol] p<500 μm when the density (ρp) is 1.4<ρp<4 g/cm3, and preferably 60 μm< [symbol] p<500 μm when the density (ρp) is 4 g/cm3 and 250 μm< [symbol] p<100 μm when the density (ρp) is 1 g/cm3 .

The oxygen-carrier material may include one or more metal oxides. According to one or more embodiments, the one or more metal oxides may be a redox-active metal oxide or a mixture of redox-active metal oxides. The redox-active metal oxide includes binary, ternary, or other mixed metal oxides capable of undergoing reduction in the presence of a reducing agent (for example, hydrogen) and oxidation in the presence of oxidizing agent (for example, oxygen or air). In some embodiments, the redox-active metal oxide may be a metal MOₓ, where M may be one or more metals of IUPAC group 6, 7, 8, 9, 10, 11, or 12 and “x” is the number of associated oxygen atoms in the structure. For example the redox-active metal oxide may be Mn₂O₃, Fe₂O₃, Co₃O₄, CuO, (LaSr)CoO₃, (LaSr)MnO₃, Mg₆MnO₈, MgMnO₃, MnO₂, Fe₃O₄, Mn₃O₄, Cu₂O, NiO, Ni₂O₃, CrO, Cr₂O₃, CrO₂, ZnO, or any combination of other IUPAC group 6-12 metal oxide. In some embodiments, the redox-active metal oxide may be cerium oxide. For example, the redox-active metal oxide may be CeO₂, Ce₂O₃, or any other mixed metal oxide containing cerium. In further embodiments, the oxygen carrier material may include lanthanum oxide, La₂O₃, in combination with other reducible metal oxides. In some embodiments, the redox-active metal oxide may be chosen from Mn₂O₃, FezO₃, CO₃O₄, CuO, (LaSr)CoO₃, (LaSr)MnO₃, Mg₆MnO₈, MgMnO₃, MnO₂, Fe₃O₄, Mn₃O₄, and Cu₂O. In some embodiments, the oxygen-carrier material may be a solid. In specific embodiments, the oxygen-carrier material may be a crushed solid or powder. In other embodiments, the oxygen-carrier material may be formulate using a redox-active metal oxide and a binder and/or support material to produce a fluidizable material with the require physical properties, for example, particle size distribution, density, and attrition resistance. The binder and/or support material may include alumina, silica, titania, magnesia, zirconia, or combinations thereof.

In one or more embodiments, the oxygen-carrier material may include a hydrogen-selective oxygen-carrier material that may include a promoter or a combination of various promoters. The addition of a promoter(s) may lead to the formation of a core-shell morphology. The promoter(s) may include alkali or alkaline-earth metal oxides from IUPAC group 1 and 2 and/or compounds comprising alkali-transition metal oxides or alkaline-earth transition metal oxides. In some embodiments, alkali elements may include one or more of sodium, lithium, potassium, and cesium. In some embodiments, alkaline-earth elements may include one or more of calcium, magnesium, strontium, and barium. In some embodiments, transition metals may include one or more of tungsten and molybdenum. For example, the one or more alkali or alkaline-earth transition metal oxides may be Na₂WO₄, K₂MOO₄, Na₂MoO₄, K₂WO₄, Li₂WO₄, CsWO₄, Li₂MoO₄, CaWO₄, CaMoO₄, MgWO₄, MgMoO₄, SrWO₄, SrMoO₄, BaWO₄ and BaMoO₄. In some embodiments, the promoter may include one or more of alkali or alkaline-earth metal salts selected from Group 1 and 2 metal cations and a counterion. In some embodiments, alkali elements may include one or more of sodium, lithium, potassium, and cesium. In some embodiments, alkaline-earth elements may include one or more of calcium, magnesium, strontium, and barium. In some embodiments, the counterion may include carbonates, sulphates, sulphites, sulfides, phosphates, phosphites and borates. For example, the alkali or alkaline-earth metal salts may be Na₂CO₃, Na₂SO₄, Na₃PO₄, Li₂CO₃, Li₂SO₄, Li₃PO₄, K2CO₃, K_{2S}O₄, K₃PO₄, Cs₂CO₃, Cs₂SO₄, Cs₃PO₄, CaCO₃, CaSO₄, Ca₃(PO₄)₂, SrCO₃, SrSO₄, Sr₃(PO₄)₂, MgCO₃, MgSO₄, Mg₃(PO₄)₂, BaCO₃, BaSO₄, Ba₃(PO₄)₂, Na₂HPO₄, KHSO₄, Na₂SO₃, K₂B₄O₇, Na₃BO₃, or combinations thereof.

For example, oxygen-carrier materials such as those disclosed in U.S. App. No. 62/725,504, entitled “METHODS OF PRODUCING HYDROGEN-SELECTIVE OXYGEN CARRIER MATERIALS,” filed on, Aug. 31, 2018, and U.S. App. No. 62/725,508, entitled “HYDROGEN-SELECTIVE OXYGEN CARRIER MATERIALS AND METHODS OF USE,” filed on, Aug. 31, 2018, are contemplated as suitable for the presently disclosed processes, and the teachings of these references are incorporated by reference herein. In one or more additional embodiments, the oxygen-carrier material may include those of U.S. Pat. No. 5,430,209, U.S. Pat. No. 7,122,495, and/or WO 2018/232133, each of which are incorporated by reference in their entireties.

The oxygen-rich oxygen-carrier material may be reducible by releasing oxygen that may be selective for combusting hydrogen. For example, the oxygen-carrier material may be selective for the combustion of hydrogen over hydrocarbons. In some embodiments, the oxygen-rich oxygen-carrier material comprises from about 1 wt. % to about 20 wt. % releasable oxygen based on total weight of the oxygen-rich oxygen-carrier material. In other embodiments, the oxygen-rich oxygen-carrier material comprises from about 1 wt. % to about 10 wt. %, from about 1 wt. % to about 5 wt. %, from about 5 wt. % to about 20 wt. %, or from about 5 wt. % to about 10 wt. % releasable oxygen. As described herein, “releasable oxygen” may refer to the oxygen that can be released through redox by the oxygen-carrier material. Other oxygen may be present in the oxygen-carrier material that is not releasable through redox. It should be understood that in some embodiments, the oxygen may be released from a surface of the oxygen-carrier material simultaneously with the combustion of hydrogen at the surface of the oxygen-carrier material.

As stated previously, the releasable oxygen of the oxygen-rich oxygen-carrier materials may be selective for combusting hydrogen over hydrocarbons. In some embodiments, at least about 60% of the releasable oxygen of the oxygen-carrier material is selective for hydrogen combustion. In other embodiments, at least about 55% of the releasable oxygen of the oxygen-carrier material is selective for hydrogen combustion.

In embodiments, when the hydrogen is contacted by the oxygen-rich oxygen-carrier material, some of the releasable oxygen is removed from the oxygen-rich oxygen-carrier material. In some embodiments, contacting the hydrogen with the oxygen-rich oxygen-carrier material removes from about 1 wt. % to 50 wt. % of the releasable oxygen from the oxygen-rich oxygen-carrier material. In other embodiments, contacting the hydrogen with the oxygen-rich oxygen-carrier material removes from about 10 wt. % to about 50 wt. %, from about 10 wt. % to about 25 wt. %, or from about 25 wt. % to about 50 wt. % of the releasable oxygen from the oxygen-rich oxygen-carrier material.

In further embodiments, when the hydrogen is contacted by the oxygen-rich oxygen-carrier material, the oxygen-rich oxygen-carrier material combusts greater than about 50% of the hydrogen. In other embodiments, when the hydrogen is contacted by the oxygen-rich oxygen-carrier material, the oxygen-rich oxygen-carrier material combusts about 50% to about 90%, or about 75% to about 90% of the hydrogen that is produced.

The contacting of the oxygen-rich oxygen-carrier material with the hydrogen may combust the hydrogen and form an oxygen-diminished oxygen-carrier material. To form the oxygen-diminished oxygen-carrier material, at least a portion of the oxygen-rich oxygen-carrier material may be reduced to a lower oxidation state. Once the oxygen-carrier material has been reduced to form the oxygen-diminished oxygen-carrier material, the oxygen-diminished oxygen-carrier material may be discharged from the fluidized bed reactor 110 at a lower oxidation state.

By way of example, ethane may be thermally dehydrogenated to from ethylene and hydrogen gas. By thermal dehydrogenation, chemicals may be dehydrogenated without the use of a dehydrogenation catalyst. Such a thermal dehydrogenation reaction scheme for the conversion of ethane to ethylene is shown in Chemical Formula 1:

C₂H₆⇔C₂H₄+H₂ ΔH=+137 kJ/mol (1)

The dehydrogenation reaction may be promoted by reducing or removing hydrogen that is produced by the thermal dehydrogenation, which pushes the reaction equilibrium toward the products. That is, in Chemical Formula 1, the removal of hydrogen pushes the equilibrium to the right, which thereby may allow the reaction to achieve increased levels of conversion or operate at lower temperatures.

The disclosed processes for the production of light olefins and aromatic olefins may incorporate thermal dehydrogenation and hydrogen combustion. Due to the removal of hydrogen by combustion with oxygen, according to one or more embodiments, the disclosed processes may operate at higher pressures and lower temperatures relative to conventional processes, yet achieve comparable conversion levels. As a result, in some embodiments the disclosed processes that incorporate hydrogen combustion may allow for relatively smaller process units and therefore reduce capital cost. It has been found that the incorporation of an oxygen-carrier material in a dehydrogenation reaction may reduce needed input heat and/or may reduce subsequent unreacted alkane, alkyl aromatic, and hydrogen separation costs. As described herein, the incorporation of an oxygen-carrier material and recycled through the process, may promote the combustion of hydrogen to form water.

On the other hand, some conventional processes for producing light olefins may require relatively high reaction temperatures. For example, some conventional processes may require reactor temperatures above 850 °C. The high temperatures may cause conventional processes to be expensive. For example, because of the higher temperatures required by these conventional processes, the reactors utilized in such processes may not have the ability to incorporate reactor internals or other design features. Alternatively, such processes may require reactor internals and other process units to be made from specialty materials, which increase capital costs.

It is contemplated that in some embodiments, combusting the formed hydrogen in the disclosed processes may simultaneously reduce the downstream separation costs. For example, in downstream processes, the product stream may require liquefaction. As such, the reduction of hydrogen in the product stream may reduce the volume of gas that would need to be liquefied or change the required temperature for liquefying the hydrocarbons due to lower hydrogen content. Therefore, the complete or partial removal of hydrogen in in the product stream may reduce the energy requirements for downstream liquefaction processes. In addition, the complete or partial removal of hydrogen in in the product stream may subsequently reduce other downstream separation costs by eliminating the need for other process units that may be utilized to separate out the hydrogen from the product stream (prior to or after liquefaction).

The production of light olefins by conventional dehydrogenation processes (e.g., those that do not incorporate hydrogen combustion) may be relatively expensive due to the high heat loads needed for the endothermic dehydrogenation reaction and/or the downstream separation steps sometimes needed to separate the unreacted alkane or alkyl aromatic and remove hydrogen that is produced in the dehydrogenation reaction. Regarding reduced heat input, catalytic dehydrogenation processes are generally endothermic and require heat. However, the exothermic combustion of hydrogen can somewhat counterbalance that heat input requirement. Additionally, the oxygen-carrier material, once diminished in oxygen content following the combustion, may be regenerated to regain its oxygen, which may be exothermic. This exothermic regeneration step may further counterbalance the heat input requirement to maintain the dehydrogenation reaction. In some embodiments, the heat produced by the oxygen-carrier regeneration and combustion reaction may completely cover the heat needed for the endothermic dehydrogenation reaction and other heat demands such as heating the feed gases (air, hydrocarbon, etc.) or balancing heat losses, or at least reduce any supplemental fuel needs of the system.

Still referring to FIG. 1, the oxygen-diminished oxygen-carrier material and the gas products may be separated within the fluidized bed reactor 110 by high efficiency cyclones. In embodiments described, the oxygen-diminished oxygen-carrier material may be passed to the regeneration unit 120 via stream 103. In further embodiments, the oxygen-carrier material may be stripped with a displacement gas such as nitrogen, steam, methane, natural gas or other suitable gas before being sent to a regeneration unit 120.

According to embodiments, the oxygen-carrier material may be passed to the regeneration unit 120 via stream 103 where regeneration occurs. Regeneration may remove the contaminant such as coke, raise the temperature of the oxygen-carrier material, or both. In some embodiments, the oxygen-diminished oxygen-carrier material may be re-oxidized to an oxidation state higher than the oxidation state of the oxygen-diminished oxygen-carrier material by combustion in an oxygen-containing environment in the regeneration unit 120. In some embodiments, the oxygen-containing environment may be air. In some embodiments of forming the oxygen-rich oxygen-carrier material, the oxygen-diminished oxygen-carrier material may be restored to its original oxidation state. In some embodiments, the oxygen-diminished oxygen-carrier material may have an oxidation state of +2, +3, or +4. The oxygen-rich oxygen-carrier material may then circulate back to the fluidized bed reactor 110, carrying the necessary heat for the dehydrogenation reaction. In other embodiments, nitrogen or steam may also be used to convey the oxygen-rich oxygen-carrier material to the fluidized bed reactor 110. The resulting gas stream from the regeneration unit 120 consists of air depleted of or containing a lower concentration of O₂.

In one or more embodiments, a supplemental fuel may be combusted in the regeneration unit 120 to produce heat and increase the temperature of the oxygen-carrier material. The heat produced by the oxidizing of the oxygen-diminished oxygen-carrier material and the combusting of the supplemental fuel may be sufficient to maintain the temperature of the fluidized bed reactor 110 at a desired temperature. The desired temperature may depend upon the minimum temperature needed for operation of the fluidized bed reactor 110, since the oxygen-carrier material may enter the fluidized bed reactor 110 and impart their temperature to the fluidized bed reactor 110.

In some embodiments, the supplemental fuel may comprise one or more of hydrogen, methane, ethane, propane, natural gas, or combinations thereof. The supplemental fuel may be gaseous. However, it should be understood that other fuel types are contemplated and within the scope the embodiments presently disclosed. The supplemental fuel may be combusted by exposure to oxygen at elevated temperatures. For example, air, oxygen enriched air, or oxygen gas may be present in the regeneration unit 120. Advantageously, in one or more embodiments, the same gas may be utilized to oxidize the oxygen-diminished oxygen-carrier material and combust the supplemental fuel.

Without being bound by theory, the amount of fuel combusted may generally be that amount which is sufficient to supply the needed to heat the fluidized bed reactor 110 where thermal dehydrogenation is taking place. While oxidation of the oxygen-diminished oxygen-carrier material may supply some heat, it may not be sufficient to heat the oxygen carrier material to a sufficient degree to heat the fluidized bed reactor 110. Therefore, the combustion of the supplemental fuel may make up the difference in heat between that utilized for the thermal dehydrogenation reaction and the oxidation of the oxygen-diminished oxygen-carrier material (as well as other reactions described herein such as water formation by hydrogen combustion).

In one or more embodiments, the regeneration unit 120 may operate at a temperature of 650 °C, or even 700 °C, to 900 °C, such as 725 °C to 875 °C, or 750 °C to 850 °C. Generally, the regeneration unit 120 may have a temperature of at least 50 °C greater than that of the fluidized bed reactor 110. Such a temperature range may be utilized so that the temperature of the fluidized bed reactor 110 may be maintained with a limited amount of oxygen-carrier material.

Still referring to FIG. 1, the oxygen-rich oxygen-carrier material may be passed from the regeneration unit 120 to the fluidized bed reactor 110 via stream 104. As such, the oxygen-carrier may be looped or recycled through the reactor system 100.

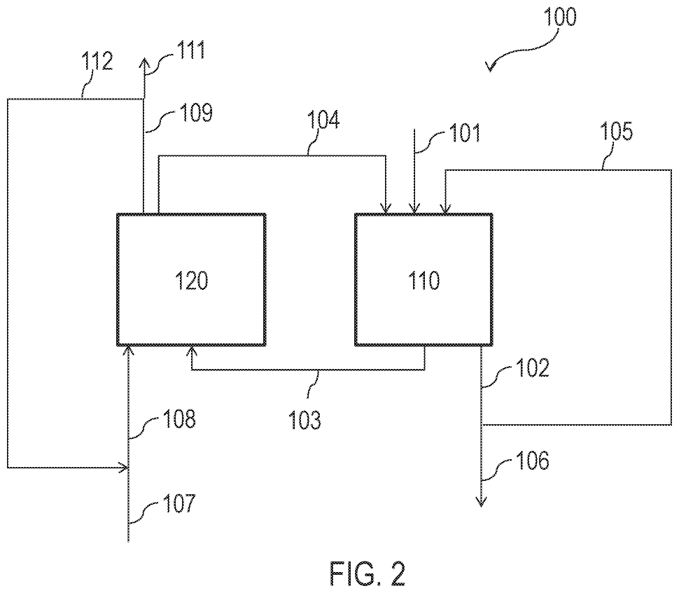

FIG. 2 depicts a system similar in many respects to that of FIG. 1, where the differences are described hereinbelow. Now referring to FIG. 2, in one or more embodiments presently described, the re-oxidation of the oxygen-diminished oxygen-carrier material may be controlled by the system depicted. For example, according to one embodiment, a flue gas may be passed into the regeneration unit 120 via stream 108. In some embodiments, the flue gas may be a recycle stream from a neighboring chemical process. In some embodiments, the oxygen-diminished oxygen-carrier material may be re-oxidized to an oxidation state higher than the oxidation state of the oxygen-diminished oxygen-carrier material by at least a portion of the flue gas exiting the regeneration unit 120 via stream 109 that is recycled to the regeneration unit 120 via stream 112. The stream 109 exiting the regeneration unit 120 may include air depleted of oxygen or containing a lower concentration of oxygen. In some embodiments, stream 112 may be mixed with fresh air via stream 107 to form stream 108. In some embodiments, stream 108 may include at least 25 mole percent (mol %) oxygen. In other embodiments, stream 108 may include from about 4 mol % to about 25 mol % oxygen, from about 4 mol % to about 21 mol %, from 4 mol % to about 10 mol % oxygen, from 10 mol % to about 25 mol % oxygen, or from 10 mol % to about 21 mol % oxygen.

In embodiments, by contacting the flue gas with the oxygen-rich oxygen-carrier material, some of the releasable oxygen is removed from the oxygen-rich oxygen-carrier material. In some embodiments, contacting the flue gas with the oxygen-rich oxygen-carrier material removes from about 0 wt. % to 15 wt. % of the releasable oxygen from the oxygen-rich oxygen-carrier material. In other embodiments, contacting the hydrogen with the oxygen-rich oxygen-carrier material removes from about 0 wt. % to about 10 wt. %, from about 0 wt. % to about 5 wt. %, or from about 5 wt. % to about 10 wt. % of the releasable oxygen from the oxygen-rich oxygen-carrier material.

In other embodiments, the oxygen-diminished oxygen-carrier material may be partially re-oxidized to an oxidation state higher than the oxidation state of the oxygen-diminished oxygen-carrier material in the regeneration unit 120. In some embodiments, the oxygen-rich oxygen-carrier material comprises less releasable oxygen than the maximum releasable oxygen capacity of the oxygen-carrier material.

In another embodiment, the oxygen-rich oxygen-carrier material may also be reduced to a lower oxidation state (“at least partially reduced”) by combusting the oxygen-rich oxygen-carrier material with a reducing gas. Without being bound by theory, in some embodiments, at least partially reducing the oxygen-rich oxygen-carrier material may precondition the oxygen-carrier material to maximize the selectivity of the fluidized bed reactor 110. The releasable oxygen bound on the surface of the oxygen-carrier material may be less selective for hydrogen combustion than the remaining bulk oxygen. In further embodiments, the oxygen-rich oxygen-carrier material may be at least partially reduced after passing from regeneration unit 120 and before passing to the fluidized bed reactor 110 in a reducer. In some embodiments, a fuel source may be used to at least partially reduce the oxygen-rich oxygen-carrier material, where a fuel source pre-combusts the oxygen that was chemically-absorbed during re-oxidation of the oxygen-diminished oxygen-carrier material in the regeneration unit 120. Depending on the configuration of the reducer, products formed by the pre-combustion may exit the reactor system 100 via a stream 102, or alternatively products formed by the pre-combustion may exit the regenerator unit 120 (such as, for example, via line 111 in FIG. 2), or at any location along line 104. In some embodiments, the products formed by the pre-combustion may be stripped from one of the process stream by, for example, nitrogen, steam or air. In some embodiments, the pre-combustion may reduce the amount of reducible oxygen on the oxygen-carrier and free oxygen between 0.01 to 10%. Without being bound by any particular theory, this oxygen is expected to be the most unselective to hydrogen combustion.

Referring now to FIGS. 1 and 2, in some embodiments, product gas from the fluidized bed reactor 110 may be passed out of the fluidized bed reactor 110 via stream 102. Stream 102 may be further processed such as by one or more subsequent separation steps or further reacted. It is contemplated that stream 102 may be utilized as a feed for another reactor system or sold as a chemical product.

In one or more embodiments, the reactor system 100 may be used to dehydrogenate hydrocarbons to produce olefins and other products (e.g., styrene from ethylbenzene), which may exit the fluidized bed reactor 110 via stream 102. In one or more embodiments, stream 102 may comprise one or more olefins and other products. Stream 102 may comprise one or more of ethylene, propylene, butylene, or styrene. According to one or more embodiments, stream 102 may comprise at least 50 wt. %, at least 60 wt. %, at least 70 wt. %, at least 80 wt. %, at least 90 wt. %, at least 95 wt. % or even at least 99 wt. % of ethylene. In additional embodiments, stream 102 may comprise at least 50 wt. %, at least 60 wt. %, at least 70 wt. %, at least 80 wt. %, at least 90 wt. %, at least 95 wt. % or even at least 99 wt. % of propylene. In additional embodiments, stream 102 may comprise at least 50 wt. %, at least 60 wt. %, at least 70 wt. %, at least 80 wt. %, at least 90 wt. %, at least 95 wt. % or even at least 99 wt. % of the sum of ethylene and propylene.

In one or more embodiments, heat gained or lost through the thermal dehydrogenation reaction, the re-oxidation of the oxygen-diminished oxygen-carrier material, and the reduction of the oxygen-rich oxygen-carrier material may create or use heat (i.e., be exothermic or endothermic). In one or more embodiments, the thermal dehydrogenation may be endothermic and results in a dehydrogenation heat loss. In some embodiments, the contacting of the hydrogen with the oxygen-rich oxygen-carrier material may be exothermic and results in a combustion heat gain. The re-oxidizing of the oxygen-diminished oxygen-carrier material may be exothermic and results in an oxygenation heat gain. As such, by incorporating hydrogen combustion during thermal dehydrogenation, in some embodiments, enough heat may be generated during the re-oxidation of the oxygen-diminished oxygen-carrier material to act as a source of heat for the alkane to olefin reaction. As such, embodiments of the disclosed process may allow for higher alkane conversion while reducing or eliminating needs for fuel gas, as may be needed for conventional cracking and/or dehydrogenation because the heat gained throughout the process by the re-oxidizing of the oxygen-carrier material, the combustion of hydrogen, or both may produce the amount of heat required for the alkanes or alkyl aromatics to olefins reaction.

Now referring to embodiments of the process depicted in FIG. 2, stream 102 or a portion of stream 102 may be passed back to the fluidized bed reactor 110 via product recycle stream 105. In some embodiments, stream 102 may include one or more unreacted alkanes or alkyl aromatics. In further embodiments, the one or more unreacted alkanes or alkyl aromatics may be passed out of the fluidized bed reactor 110 to a separation unit (not pictured) via stream 102. The one or more unreacted alkanes or alkyl aromatics may be separated from a remainder of the dehydrogenation effluent using the separation unit. In some embodiments, the one or more unreacted alkanes or alkyl aromatics may then be transported out of the separation unit and passed to the fluidized bed reactor 110 via product recycle stream 105. In some embodiments from about 10% to about 90% of the one or more unreacted alkanes or alkyl aromatics may be passed via product recycle stream 105 to the fluidized bed reactor 110. In other embodiments, from about 20% to about 90%, from about 30% to about 90%, from about 40% to about 90%, from about 50% to about 90%, from about 60% to about 90% from about 70% to about 90%, or from about 80% to about 90% of the one or more unreacted alkanes or alkyl aromatics may be passed via product recycle stream 105 to the fluidized bed reactor 110.

The present disclosure includes several aspects. In a first aspect of the present disclosure a method for dehydrogenating hydrocarbons may include passing a hydrocarbon feed comprising one or more alkanes or alkyl aromatics into a fluidized bed reactor. In the fluidized bed reactor at least 95 wt. % of the hydrocarbon feed has an atmospheric boiling point of less than or equal to 300 °C. The method further comprises thermally cracking the hydrocarbon feed in the fluidized bed reactor to produce a dehydrogenated product and hydrogen. The fluidized bed reactor operates at a temperature of at least 600 °C. The fluidized bed reactor is free of dehydrogenation catalyst. The method further comprises contacting the hydrogen with an oxygen-carrier material in the fluidized bed reactor to combust the hydrogen and form an oxygen-diminished oxygen-carrier material. The oxygen-carrier material is reducible. The method further comprises passing the oxygen-diminished oxygen-carrier material to a regeneration unit, oxidizing the oxygen-diminished oxygen-carrier material in the regeneration unit to form the oxygen-rich oxygen-carrier material, combusting a supplemental fuel in the regeneration unit to produce heat and increase the temperature of the oxygen-carrier material, and passing the oxygen-rich oxygen-carrier material to the fluidized bed reactor.

A second aspect of the present disclosure includes the first aspect, further comprising partially reducing the oxygen-rich oxygen-carrier material prior to contacting the hydrogen with the oxygen-rich oxygen-carrier material in the fluidized bed reactor.

A third aspect of the present disclosure includes any of the previous aspects, where the fluidized bed reactor operates at a temperature of at least 600 °C and less than 850 °C.

A fourth aspect of the present disclosure includes any of the previous aspects, where the supplemental fuel is chosen from hydrogen, methane, ethane, propane, natural gas, or combinations thereof.

A fifth aspect of the present disclosure includes any of the previous aspects, where all solid particulate materials in the fluidized bed reactor is oxygen-carrier material.

A sixth aspect of the present disclosure includes any of the previous aspects, where the oxygen-rich oxygen-carrier material comprises from 1 wt. % to 20 wt. % releasable oxygen based on total weight of the oxygen-rich oxygen-carrier material.

A seventh aspect of the present disclosure includes any of the previous aspects, where contacting the hydrogen with the oxygen-rich oxygen-carrier material removes from 1 wt. % to 50 wt. % of the releasable oxygen from the oxygen-rich oxygen-carrier material.

An eighth aspect of the present disclosure includes any of the previous aspects, where contacting the hydrogen with the oxygen-rich oxygen-carrier material combusts greater than 50% of the hydrogen.

A ninth aspect of the present disclosure includes any of the previous aspects, where the oxygen-carrier material comprises one or more metal oxides.

A tenth aspect of the present disclosure includes any of the previous aspects, where the oxygen-carrier material exhibits Geldart A or Geldart B properties.

It will be apparent to those skilled in the art that various modifications and variations can be made to the presently disclosed technology without departing from the spirit and scope of the technology. Since modifications combinations, sub-combinations and variations of the disclosed embodiments incorporating the spirit and substance of the presently disclosed technology may occur to persons skilled in the art, the technology should be construed to include everything within the scope of the appended claims and their equivalents. Additionally, although some aspects of the present disclosure may be identified herein as preferred or particularly advantageous, it is contemplated that the present disclosure is not limited to these aspects.

It is noted that the various details described in this disclosure should not be taken to imply that these details relate to elements that are essential components of the various embodiments described in this disclosure, even in cases where a particular element is illustrated in each of the drawings that accompany the present description. Unless specifically identified as such, no feature disclosed and described herein should be construed as “essential”. Contemplated embodiments of the present technology include those that include some or all of the features of the appended claims.

For the purposes of describing and defining the present disclosure it is noted that the term “about” are utilized in this disclosure to represent the inherent degree of uncertainty that may be attributed to any quantitative comparison, value, measurement, or other representation. The term “about” are also utilized in this disclosure to represent the degree by which a quantitative representation may vary from a stated reference without resulting in a change in the basic function of the subject matter at issue.

In relevant cases, where a composition is described as “comprising” one or more elements, embodiments of that composition “consisting of” or “consisting essentially of” those one or more elements is contemplated herein.

In some embodiments, chemicals or chemical streams are described as “passing” from one system unit or portion of a system unit to another. As described herein, such passing may include direct passing or indirect passing. For example, when passing from “unit A” to “unit B”, direct passing has no intermediate destination between unit A and unit B (i.e., directly through a pipe or other transport passage), and indirect passing may include one or more intermediate destinations between unit A and unit B. For example, a stream passing from unit A to unit B may passed through, without limitation, a heat exchanger, treatment device, etc.

It should be appreciated that compositional ranges of a chemical constituent in a stream or in a reactor should be appreciated as containing, in some embodiments, a mixture of isomers of that constituent. For example, a compositional range specifying butene may include a mixture of various isomers of butene. It should be appreciated that the examples supply compositional ranges for various streams, and that the total amount of isomers of a particular chemical composition can constitute a range.

It is noted that one or more of the following claims and the detailed description utilize the terms “where” or “wherein” as a transitional phrase. For the purposes of defining the present technology, it is noted that this term is introduced in the claims as an open-ended transitional phrase that is used to introduce a recitation of a series of characteristics of the structure and should be interpreted in like manner as the more commonly used open-ended preamble term “comprising.”It should be understood that any two quantitative values assigned to a property may constitute a range of that property, and all combinations of ranges formed from all stated quantitative values of a given property are contemplated in this disclosure. Where multiple ranges for a quantitative value are provided, these ranges may be combined to form a broader range, which is contemplated in the embodiments described herein. The following listing of claims will replace all prior versions, and listings, of claims in the present application:

1. A method for dehydrogenating hydrocarbons comprising:
passing a hydrocarbon feed comprising one or more alkanes or alkyl aromatics into a fluidized bed reactor, wherein at least 95 wt. % of the hydrocarbon feed has an atmospheric boiling point of less than or equal to 300 °C;
thermally cracking the hydrocarbon feed in the fluidized bed reactor to produce a dehydrogenated product and hydrogen, wherein the fluidized bed reactor operates at a temperature of at least 600 °C, and wherein the fluidized bed reactor is free of dehydrogenation catalyst; and
contacting the hydrogen with an oxygen-rich oxygen-carrier material in the fluidized bed reactor to combust the hydrogen and form an oxygen-diminished oxygen-carrier material, wherein the oxygen-rich oxygen-carrier material is reducible;
passing the oxygen-diminished oxygen-carrier material to a regeneration unit;
oxidizing the oxygen-diminished oxygen-carrier material in the regeneration unit to form the oxygen-rich oxygen-carrier material;
combusting a supplemental fuel in the regeneration unit to produce heat and increase the temperature of the oxygen-carrier material; and
passing the oxygen-rich oxygen-carrier material to the fluidized bed reactor.

2. The method of any claim 1, further comprising partially reducing the oxygen-rich oxygen-carrier material prior to contacting the hydrogen with the oxygen-rich oxygen-carrier material in the fluidized bed reactor.

3. The method of claim 1, wherein the fluidized bed reactor operates at a temperature of at least 600 °C and less than 850 °C.

4. The method of claim 1, wherein the supplemental fuel is chosen from hydrogen, methane, ethane, propane, natural gas, or combinations thereof.

5. The method of claim 1, wherein all solid particulate materials in the fluidized bed reactor is oxygen-carrier material.

6. The method of claim 1, wherein the oxygen-rich oxygen-carrier material comprises from 1 wt. % to 20 wt. % releasable oxygen based on total weight of the oxygen-rich oxygen-carrier material.

7. The method of claim 1, wherein contacting the hydrogen with the oxygen-rich oxygen-carrier material removes from 1 wt. % to 50 wt. % of the releasable oxygen from the oxygen-rich oxygen-carrier material.

8. The method of claim 1, wherein contacting the hydrogen with the oxygen-rich oxygen-carrier material combusts greater than 50% of the hydrogen.

9. The method of claim 1, wherein the oxygen-carrier material comprises one or more metal oxides.

10. The method of claim 1, wherein the oxygen-carrier material exhibits Geldart A or Geldart B properties.
